(19) United States
(12) Patent Application Publication
(10) Pub. No.: US 2014/0066629 A1
(43) Pub. Date: Mar. 6, 2014

(54) PROCESS FOR THE PREPARATION OF AROMATIC AZOLE COMPOUNDS

(71) Applicants: MARC B. GOLDFINGER, West Chester, PA (US); TIMOTHY PELLENBARG, Wilmington, DE (US)

(72) Inventors: MARC B. GOLDFINGER, West Chester, PA (US); TIMOTHY PELLENBARG, Wilmington, DE (US)

(73) Assignee: E I DU PONT DE NEMOURS AND COMPANY, Wilmington, DE (US)

(21) Appl. No.: 13/597,988

(22) Filed: Aug. 29, 2012

(51) Int. Cl. C07D 487/04; C07D 235/18; C07D 263/57; C07D 513/04

(52) U.S. Cl. 548/151; 548/302.1; 548/310.7; 548/224

(57) ABSTRACT

Aromatic azole compounds such as 2-(4-aminophenyl)-5-amino-benzimidazole are prepared in an organic sulfonic acid solvent instead of polyphosphoric acid. This allows recovery and recycle of the solvent and avoids the handling and environmental concerns resulting from the use of polyphosphoric acid. The resulting compounds find use in the pharmaceutical industry, as anticorrosion agents, and as precursors for high-performance fibers having high strength, stiffness, and flame resistance.

### TECHNICAL FIELD

The disclosure relates to methods of making aromatic azole compounds such as 2-(4-aminophenyl)-5-amino-benzimidazole and related compounds.

### BACKGROUND

Aromatic azoles are highly useful compounds, finding application, for example, in the pharmaceutical industry, as anticorrosion agents, and as precursors for high-performance fibers having high strength, stiffness, and flame resistance.

Since the 1950's, aromatic azoles have typically been synthesized by condensation reactions of aromatic diamines and aromatic carboxylic acids in polyphosphoric acid (PPA). Attempts to use other acid systems such as sulfuric acid, phosphoric acid, hydrochloric acid resulted in fair to poor yields (D. W. Hein, et al., Journal of the American Chemical Society, 79 (1957) 427-429). PPA facilitates the condensation reaction in three ways: it is a solvent for the reagents; it is a dehydrating agent, which thus drives the condensation reaction forward; and it is an activating agent, in that it reacts with the carboxylic acid reactant to form a mixed anhydride, a more reactive moiety. However, the use of PPA is not without issues. It is highly viscous. PPA has a very low vapor pressure and thus cannot practically be recovered from the reaction mixture by distillation for recycle or disposal. Instead, it is more typically hydrolyzed to phosphoric acid. Additionally, waste disposal of phosphates poses challenges with respect to the environment, such as algal bloom.

Thus, there exists a need for a process to synthesize aromatic azole compounds in a medium that avoids the issues raised by the use of PPA.

### SUMMARY OF THE INVENTION

In one aspect, the present invention is a process for preparing a compound that is represented by the structure of the following Formula (I)

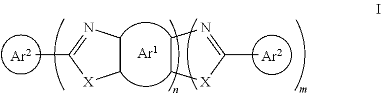

comprising the steps:
a) forming a reaction mixture comprising
   i) a stoichiometric mixture of an aromatic amine component which is an ortho-substituted aromatic amine represented by the structure of the following Formula (II),

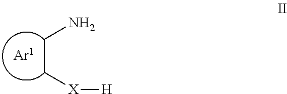

or an acid salt of such an amine;
and an aromatic acid component represented by the structure of the following Formula (III), and

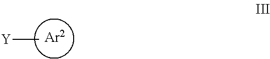

ii) a solvent comprising an organic sulfonic acid represented by the structure of the following Formula (IV); and

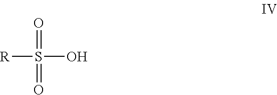

b) heating the reaction mixture at a temperature below the decomposition temperature of the organic sulfonic acid, for a time sufficient to effect a reaction to produce the composition that is represented by the structure of Formula (I);
wherein:
   each Ar¹ is a substituted or unsubstituted, monocyclic or polycyclic, arylene or heteroarylene group;
   each Ar² is a substituted or unsubstituted, monocyclic or polycyclic, aryl or heteroaryl group;
   n=1 or 2 and m=0 or 1;
   each X is independently NH, O, or S;
   each Y is independently COOH, COOR′, COHal, CONH₂, or CN; and
   R and each R′ are each independently an alkyl, aryl, alkaryl, or aralkyl group and Hal is a halogen atom.

### DETAILED DESCRIPTION

The methods described herein are described with reference to the following terms.

As used herein, where the indefinite article “a” or “an” is used with respect to a statement or description of the presence of a step in a process of this invention, it is to be understood, unless the statement or description explicitly provides to the contrary, that the use of such indefinite article does not limit the presence of the step in the process to one in number.

As used herein, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the term “invention” or “present invention is a non-limiting term and is not intended to refer to any single variation of the particular invention but encompasses all possible variations described in the specification and recited in the claims.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. The term “about” may mean within 10% of the reported numerical value, preferably within 5% of the reported numerical value.

As used herein, the term “alkyl” is used to denote a univalent group derived from an alkane by removing a hydrogen atom from any carbon atom: —CₙH₂ₙ₊₁ where n≧1; as used herein, the term “alkyl” includes both substituted and unsubstituted groups.

As used herein, the term “aryl” is used to denote a univalent group whose free bonding site is to a carbon atom of an aromatic ring; as used herein, the term “aryl” includes both substituted and unsubstituted groups. An example is the phenyl group, i.e., the C₆H₅ radical shown below:

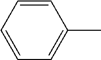

In a “heteroaryl” radical one or more O, N, or S atoms are substituted for any one or more of the in-ring carbon atoms of an aryl radical, provided that the resulting structure contains no —O—O— or —S—S— moieties. As used herein, the term “heteroaryl” includes both substituted and unsubstituted groups. An example is the 4-pyridyl group, i.e., the C₅H₄N radical shown below:

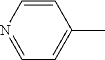

As used herein, the term “aralkyl” denotes an alkyl group which bears an aryl group; as used herein, the term “aralkyl” includes both substituted and unsubstituted groups. One such example is the benzyl group, i.e., the C₇H₇ radical shown below:

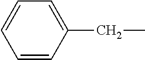

As used herein, the term “alkaryl” denotes an aryl group which bears an alkyl group; as used herein, the term “alkaryl” includes both substituted and unsubstituted groups. One example of an alkaryl group is the 4-methylphenyl radical, C₇H₇, shown below:

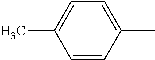

As used herein, the term “arylene” denotes a multivalent aromatic radical formed by the removal of two or more hydrogens from different carbon atoms on the aromatic ring, or on the aromatic rings when the structure is polycyclic. In a “heteroarylene” radical, one or more O, N, or S atoms are substituted for any one or more of the in-ring carbon atoms, provided that the resulting structure contains no —O—O— or —S—S— moieties.

As used herein, the term “aromatic azole compound” denotes a compound that contains an unsaturated 5-membered ring which contains at least two heteroatoms, at least one of which is nitrogen; and an aromatic moiety to which the 5-membered ring is fused or otherwise bonded. The rings may carry substituents. A few aromatic azole structures are shown below for illustration:

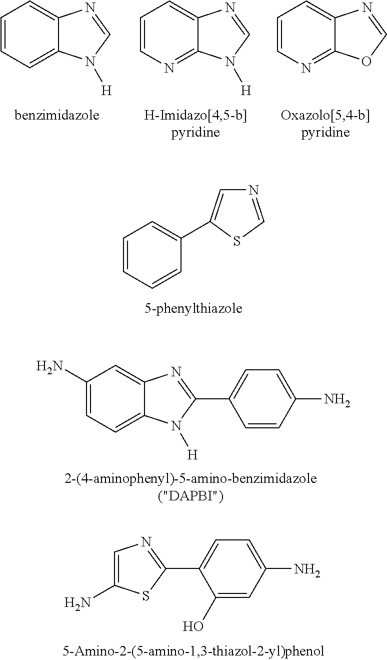

As used herein, the term “stoichiometric ratio” for a given reaction is the molar ratio of reagents for which, on completion, the reaction would have consumed all the reagents completely and left no residue. The term “stoichiometric ratio” allows for small deviations from strict stoichiometry, as may be the case, for example, when a small amount of the less expensive ingredient is used in a slight excess (e.g., a few mole percent) for economy and to aid in driving the reaction forward.

As used herein, the term “organic” denotes carbon-containing compounds with the following exceptions: binary compounds as the carbon oxides, carbides, carbon disulfide, etc.; ternary compounds such as metallic cyanides, metallic carbonyls, phosgene, carbonyl sulfide; and metallic carbonates such as calcium carbonate and sodium carbonate.

As used herein, the term “acid salt” of a specified amine refers to a salt formed by reaction of an organic acid or a mineral acid with the amine. As used herein, the term “organic acid” means an organic compound having acidic properties; some examples are acetic acid, formic acid, and methane sulfonic acid. As used herein, the term “mineral acid” means an inorganic acid, as distinguished from organic acid. Some examples are sulfuric acid, nitric acid, phosphoric acid, and hydrochloric acid.

Features of certain of the processes of this invention are described herein in the context of one or more specific embodiments that combine various such features together. The scope of the invention is not, however, limited by the description of only certain features within any specific embodiment, and the invention also includes (1) a subcombination of fewer than all of the features of any described embodiment, which subcombination may be characterized by the absence of the features omitted to form the subcombination; (2) each of the features, individually, included within the combination of any described embodiment; and (3) other combinations of features formed by grouping only selected features taken from two or more described embodiments, optionally together with other features as disclosed elsewhere herein. Some of the specific embodiments of the processes hereof are as follows:

One embodiment of this invention provides a process for preparing a compound that is represented by the structure of the following Formula (I)

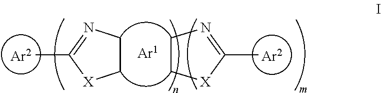

comprising the steps:
a) forming a reaction mixture comprising
   i) a stoichiometric mixture of an aromatic amine component which is an ortho-substituted aromatic amine represented by the structure of the following Formula (II),

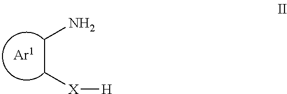

or an acid salt of such an amine;
and an aromatic acid component represented by the structure of the following Formula (III), and

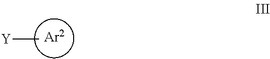

ii) a solvent comprising an organic sulfonic acid represented by the structure of the following Formula (IV); and

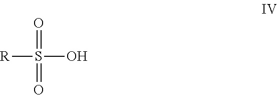

b) heating the reaction mixture at a temperature below the decomposition temperature of the organic sulfonic acid, for a time sufficient to effect a reaction to produce the composition that is represented by the structure of Formula (I);
wherein:
   each Ar¹ is a substituted or unsubstituted, monocyclic or polycyclic, arylene or heteroarylene group;
   each Ar² is a substituted or unsubstituted, monocyclic or polycyclic, aryl or heteroaryl group;
   n=1 or 2 and m=0 or 1;
   each X is independently NH, O, or S;
   each Y is independently COOH, COOR′, COHal, CONH₂, or CN; and
   R and each R′ are each independently an alkyl, aryl, alkaryl, or aralkyl group.

It is well known that, when X is NH, the imidazole ring thereby produced exists in two equivalent tautomeric forms:

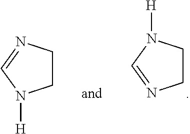 [SMILES: C1=NCCN1 ; C1=NCCN1]

Thus, equivalent structures of Formula (I) wherein X═NH are:

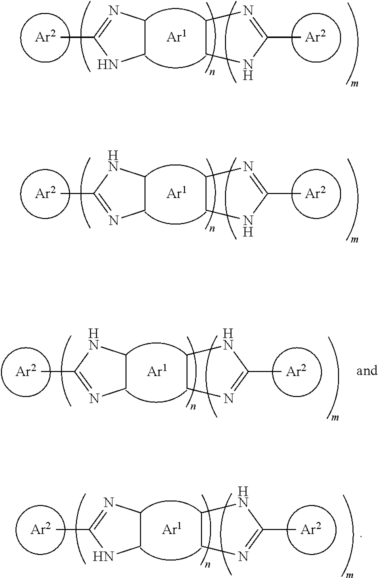

If, in Formula (I), both m and n are nonzero and X is O (oxazole) or S (thiazole), the structure is fixed and determined by the structure of the starting monomer. For example, to make

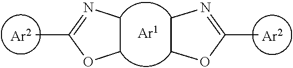

(n=1, m=1, each X=0) using the process described herein, the compound of Formula (II) would be

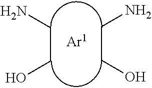

(for example, 1,3-diamino-4,6-dihydroxy-benzene). To make

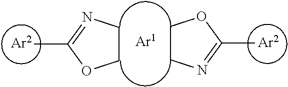

using the process described herein, the compound of Formula (II) instead would be

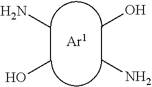

(for example, 1,4-diamino-2,5-dihydroxy-benzene).

Examples of ortho-substituted aromatic amines described by Formula (II), suitable for use in the processes described herein, include without limitation: 1,2-diaminobenzene, 1,2,4-triaminobenzene; 1,2,4,5-tetraminobenzene; 1,2-diaminopyridine, 1,2,4-triaminopyridine, 2,3,5,6-tetraminopyridine; 2,4-diaminophenol; 2-aminophenol; 2-aminothiophenol; 1,3-diamino-4,6-dihydroxybenzene; 1,4-diamino-2,5-dihydroxybenzene; 2,5-diaminobenzene-1,4-dithiol; and their acid salts. The aromatic amine component can be a mixture of at least two aromatic amines or of their acid salts, e.g., salts of the ortho-substituted aromatic amines with acetic acid, HCl, H₂SO₄, H₃PO₄, or sulfonic acids.

The compound described by Formula (III) is referred herein as the aromatic acid component and can be an aromatic acid itself (Y═COOH) or an ester (Y═COOR′), amide (Y═CONH₂), acid halide (Y═COHal, where “Hal” is a halogen atom such as Cl, Br or I, for example), or nitrile derivative (Y═CN). Mixtures of compounds described by Formula (III) can also be used as the aromatic acid component. Ar² can bear substituents in addition to Y. Examples of compounds suitable for use as the aromatic acid component in the processes described herein include without limitation: benzoic acid, halo-substituted benzoic acids, alkyl-substituted benzoic acids, 4-aminobenzoic acid, 3-aminobenzoic acid, 2-aminobenzoic acid, terephthalic acid, isophthalic acid, benzene-1,2-dicarboxylic acid, terephthaloyl chloride, isophthaloyl chloride, benzonitrile, benzamide, alkyl benzoates, and mixtures of at least two of these.

In this process, the aromatic amine and aromatic acid components are provided in a stoichiometric ratio, the ratio depending on what, where, and how many reactive groups are present. As used herein, the term “stoichiometric ratio” allows for small deviations (less than a few mole percent) from strict stoichiometry, as may be the case, for example, when a small amount of the less expensive ingredient is used in a slight excess for economy and to aid in driving the reaction forward.

In one embodiment, the stoichiometric ratio is 1:1. In this embodiment, n=1 and m=0, and Formula (I) becomes:

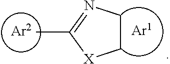

In an example of this embodiment, Ar¹ is 3-amino-o-phenylene, C₆H₃NH₂,

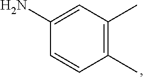

Ar² is 4-aminophenyl, C₆H₄NH₂,

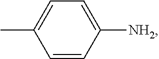

Y is COOH, X is NH, R is CH₃, and the compound of Formula (I) thereby produced, having n=1 and m=0, is 2-(4-aminophenyl)-5-amino-benzimidazole (“DAPBI”):

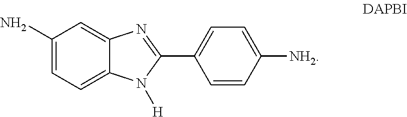 [SMILES: Nc1ccc(-c2nc3cc(N)ccc3[nH]2)cc1]

In another embodiment, the stoichiometric ratio of aromatic acid component to aromatic amine component is 2:1. In this embodiment, n=m=1 and Formula (I) becomes:

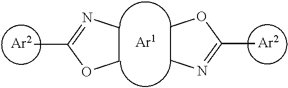

In an example of this embodiment, one mole of 1,2,4,5-tetraminobenzene can react with two moles of p-aminobenzoic acid. Here, Ar¹ is 3,4-diamino-o-phenylene,

### C₆H₂ (NH₂)₂,

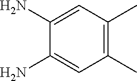

Ar² is 4-aminophenyl (C₆H₄NH₂),

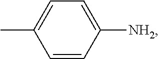

Y is COOH, X is NH, R is CH₃, and the compound of Formula (I) thereby produced is:

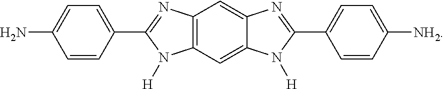 [SMILES: Nc1ccc(-c2nc3cc4nc(-c5ccc(N)cc5)[nH]c4cc3[nH]2)cc1]

In another embodiment, the stoichiometric ratio of aromatic acid component to aromatic amine component is 1:2. In this embodiment, n=2, m=0, and Formula (I) becomes:

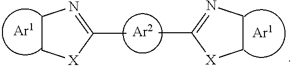

In an example of this embodiment, one mole of terephthalic acid can react with two moles of 1,2,4-triaminobenzene. Ar¹ is 3-amino-o-phenylene, C₆H₃NH₂,

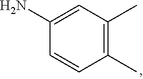

Ar² is p-C₆H₄COOH,

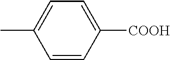

Y is COOH, X is NH, R is CH₃, and the compound of Formula (I) thereby produced is:

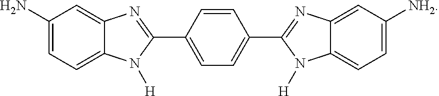 [SMILES: Nc1ccc2[nH]c(-c3ccc(-c4nc5cc(N)ccc5[nH]4)cc3)nc2c1]

When mixtures of ortho-substituted amines and/or mixtures of aromatic acid components are used, the aromatic acid to aromatic amine stoichiometric ratio can be a non-integer, depending on the specific compounds used, and is readily determined by one of ordinary skill in the art.

The reaction mixture includes an organic sulfonic acid (IV),

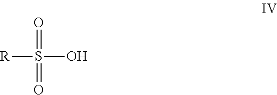

wherein R is an alkyl, aryl, alkaryl, or aralkyl group. In an embodiment, R is a C₁₋₁₈ alkyl group. In an embodiment, R is methyl; i.e., the organic sulfonic acid is methanesulfonic acid (“MsOH”). An added benefit is that MsOH is biodegradable (MSA™ methanesulfonic acid product literature, 2005, Arkema Inc., Philadelphia, Pa., USA). In an embodiment, R is C₇H₇,

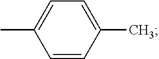

i.e., the organic sulfonic acid is p-toluenesulfonic acid.

The aromatic amine component and aromatic acid component together are present in the reaction mixture at about 1 to about 30 weight percent, based on the combined weight of aromatic amine, aromatic acid, and organic sulfonic acid. In an embodiment, the aromatic amine component and the aromatic acid component together are present at about 15 to 17 weight percent. In some embodiments, the aromatic amine component and aromatic acid component together are present at a weight percentage between and optionally including any two of the following values: 1, 3, 5, 7, 9, 11, 13, 15, 17, 19, 21, 23, 25, 27, 29, and 30 weight percent. Higher concentrations of aromatic diamine component and aromatic acid component can lead to undesirable side reactions.

It can be advantageous to add a reducing agent to the reaction mixture to prevent oxidation of the aromatic diamine component. The reducing agent is capable of reducing oxidation byproducts at the pH of the reaction mixture in which it is present. Such reducing agents are effective at both eliminating and preventing formation of oxidation byproducts. It is preferred that the reducing agents, when oxidized, do not react with any species present to form undesirable insoluble byproducts. Examples of suitable reducing agents for the reaction mixtures described herein include but are not limited to Sn(0), Sn(II), Cr(II), Mn(II), Fe(0), Fe(II), Co(0), Co(II), Ni(0), Ni(II), Cu(0), Cu(I), Zn(0), Mg(0), and mixtures thereof. In an embodiment, the reducing agent is Sn(II). These reducing agents are typically used in an amount between about 0.1 wt % and about 10 wt %. In an embodiment, the reducing agent is used in an amount between about 0.1 wt % and about 5 wt %. In an embodiment, the reducing agent is used in an amount between about 0.5 wt % and about 3.5 wt %. In some embodiments, the reducing agent is used in an amount between and optionally including any two of the following values: 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, and 10.0 wt %.

The reaction mixture is heated at a reaction temperature below the decomposition temperature of the organic sulfonic acid, for a time sufficient to effect a reaction to produce the composition that is represented by the structure of Formula (I). Methanesulfonic acid starts to decompose at about 180° C.; thus, in an embodiment, the sulfonic acid is methanesulfonic acid and the reaction temperature is between about 100° C. and about 170° C., in a further embodiment between about 150° C. and about 165° C. Appropriate reaction times and temperatures depend on the individual system and are readily determined by one of ordinary skill in the art.

In another embodiment, the reaction mixture further comprises a solvent that is immiscible with the organic sulfonic acid under reaction conditions, boils between 100° C. and 200° C., and forms an azeotrope with water produced by the reaction; wherein the process further comprises the step of removing said azeotrope from the reaction mixture by distillation. When such an azeotroping solvent is present, a biphasic mixture is formed in the reaction vessel. The lower layer of the biphasic mixture contains the reaction mixture: aromatic amine component, aromatic acid component, organic sulfonic acid, and, if used, reducing agent. The upper layer contains the azeotroping solvent. The azeotrope formed by the azeotroping solvent and the water of reaction is distilled off, removing water and thus driving the reaction forward. Examples of suitable azeotroping solvents include without limitation: xylenes, toluene, octane, chlorobenzenes, and alcohols (e.g., 1-butanol), though some of the alcohol may be converted to the corresponding diether compound. In some embodiments, the azeotroping solvent is p-xylene; in another, octane. Enough azeotroping solvent is added to cover the surface of the lower layer. In some embodiments, the azeotroping solvent is present at about 0.5 to about 2 times the volume of the lower layer.

The present invention allows aromatic azole compounds such as 2-(4-aminophenyl)-5-amino-benzimidazole to be prepared in an organic sulfonic acid solvent instead of polyphosphoric acid. This allows recovery and recycle of the solvent and avoids the handling and environmental concerns resulting from the use of polyphosphoric acid. The resulting compounds find use in the pharmaceutical industry, as anticorrosion agents, and as precursors for high-performance fibers having high strength, stiffness, and flame resistance.

### EXAMPLES

The advantageous attributes and effects of the processes hereof may be seen in a series of examples as described below. The embodiments of these processes on which the examples are based are representative only, and the selection of those embodiments to illustrate the invention does not indicate that materials, reactants, conditions, steps, techniques, or protocols not described in these examples are not suitable for practicing these processes, or that subject matter not described in these examples is excluded from the scope of the appended claims and equivalents thereof.

### Materials

Degussa F101 catalyst, dry basis 5% Pt on carbon (“Pt/C”) catalyst, wetted with 50% water, was obtained from Degussa, now Evonik Degussa, a subsidiary of Evonik Industries AG, Essen, Germany.
2,5-Diaminobenzene-1,4-dithiol dihydrochloride (minimum 95% purity) and 4-aminobenzoic acid were obtained from TCI America (Portland, Oreg., USA).
Methanesulfonic acid (>99.5% purity), 2,4-diaminophenol dihydrochloride (98% purity), and 2,4-dinitroaniline (98% purity) were obtained from Sigma-Aldrich Corporation (St. Louis, Mo., USA).
1,3-Diamino-4,6-dinitrobenzene was synthesized as described in U.S. Pat. No. 8,071,812.
1,2,4-triaminobenzene dihydrochloride and 1,2,4,5-tetraminobenzene tetrahydrochloride were synthesized as follows:

### 1,2,4-triaminobenzene dihydrochloride

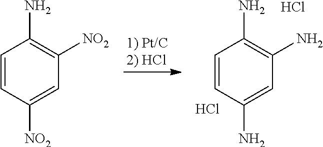 [SMILES: Nc1ccc([N+](=O)[O-])cc1[N+](=O)[O-] ; Cl ; Nc1ccc(N)c(N)c1 ; Cl]

1,2,4-triaminobenzene and its mineral and organic acid salts are susceptible to oxidation and should be handled in an inert atmosphere. To a 2 gallon autoclave was added 350 g of 2,4-dinitroaniline and 7.0 g of Degussa F101 Pt/C catalyst. The clave was then sealed, purged with nitrogen, and had added to it 1750 mL of nitrogen sparged ethanol. The clave was heated to 70° C. and pressurized to 100 psi with hydrogen and then kept at 70° C. and 100 psi for 2 h. After 2 h the claves contents were pushed through a solids filter and into a precipitation vessel where they were cooled to 15° C. and the product precipitated with the addition 595 mL of 12.1M HCl. The precipitated solids were collected, washed with 12.1M HCl, and partially dried on the filter at 65° C. with nitrogen and vacuum. This afforded 362.3 g of wet (with ethanol) 1,2,4-triaminobenzene dihydrochloride.

The 1,2,4-triaminobenzene dihydrochloride was further purified by crystallization from aqueous with concentrated hydrochloric acid. From the 362.3 g of the crude (wet with ethanol) 1,2,4-triaminobenzene dihydrochloride, 315.42 g dried, crystallized product was recovered, or 78% yield from the hydrogenation.

### 1,2,4,5-tetraminobenzene tetrahydrochloride

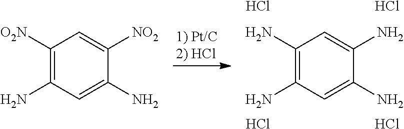 [SMILES: Cl ; Cl ; Nc1cc(N)c([N+](=O)[O-])cc1[N+](=O)[O-] ; Nc1cc(N)c(N)cc1N ; Cl ; Cl]

1,2,4,5-tetraminobenzene and its mineral and organic acid salts are susceptible to oxidation and should be handled in an inert atmosphere. To a 2 gallon autoclave was added 480 g of 1,5-diamino-2,4-dinitrobenzene, 5.5 g of T\tin powder, and 9.6 g of Degussa F101 Pt/C catalyst. The clave was then sealed, purged with nitrogen, and had added to it 2000 mL of nitrogen sparged ethanol. The clave was heated to 70° C. and pressurized to 300 psi with hydrogen and then kept at 80.5° C. and 300 psi for 2 h. After 2 h, the claves contents were pushed through a solids filter and into a precipitation vessel where they were cooled to 15° C. and the product precipitated with the addition 1400 mL of 12.1M HCl. The precipitated solids were collected, washed with 12.1 M HCl and ethanol, and partially dried on the filter at 40° C. with nitrogen and vacuum. This afforded 557.3 g (81% yield) of 1,2,4,5-tetraminobenzene tetrahydrochloride.

The 1,2,4,5-tetraminobenzene tetrahydrochloride can be further purified by crystallization from aqueous with concentrated hydrochloric acid.

### ABBREVIATIONS

In the examples, the meaning of abbreviations is as follows: “g” means gram(s), “¹H NMR” means proton nuclear magnetic resonance spectroscopy, “h” means hour(s), “L” means liter(s), “mg” means milligram(s) “mL” means milliliter(s), “min” means minutes, “M” means molar, and “MsOH” means methanesulfonic acid.

### Example 1

This example demonstrates the synthesis of 2-(4-aminophenyl)-5-amino-benzimidazole from 1,2,4-triaminobenzene dihydrochloride and 4-aminobenzoic acid in methanesulfonic acid.

To a round bottomed flask was added 700 mg of SnCl₂ dihydrate, 16.79 g 1,2,4-triaminobenzene dihydrochloride, 9.19 g 4-aminobenzoic acid, and 60 mL of methanesulfonic acid. The stirred solution was slowly heated to 160-165° C. The reaction was continuously swept with nitrogen. The reaction was taken off heat at 2 h 40 min.

The reaction solution was diluted with 180 mL of water and heated to 90-95° C. and 37 mL of 12.1 M HCl was slowly added. The solution was cooled, providing solids that were isolated by filtration and washed with fresh 12.1 M HCl. A second crop of solids was obtained on adding additional HCl to the filtrate. The solids were combined and dissolved in 400 mL hot water. The solution was brought to 90-95° C. and had added to it 2.0 g Darco 100 mesh activated carbon. The solution was stirred for 30 minutes at this temperature and then the activated carbon was removed by filtration. While the solution was still hot (70° C.), 10.0 M NaOH was added until the pH was adjusted to ˜10 causing solids to precipitate. The solution was then allowed to cool to room temperature and the solids were collected by filtration, washed with 2×50 mL water with mixing and then dried overnight in a vacuum oven at 70° C. An off-white solid (14.6 g) was recovered. The structure of the product was confirmed by ¹H NMR and Mass Spectrometry.

### Example 2

This example demonstrates the synthesis of 2-(4-aminophenyl)-1,3-benzoxazol-5-amine from 2,4-diaminophenol dihydrochloride and p-aminobenzoic acid in methanesulfonic acid.

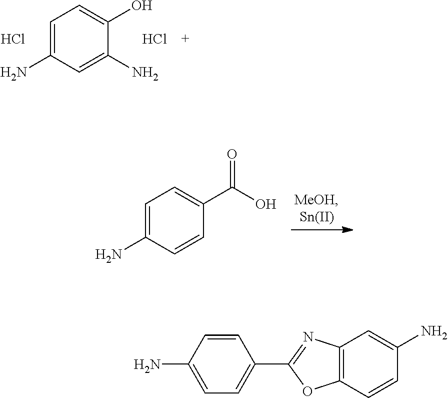

In a nitrogen filled glovebox to a 100 mL round bottomed Schlenk flask equipped with a magnetic stir bar was added 172 mg of SnCl₂ dihydrate, 5.00 g 2,4-diaminophenol dihydrochloride, 3.48 g p-aminobenzoic acid, and 22.7 mL of methanesulfonic acid. Methanesulfonic acid addition would cause HCl outgassing which was captured via a sodium carbonate bath. The flask was purged with nitrogen and then lowered into a hot oil bath whose temp was 75° C. The oil bath temp was held at 75° C. until HCl gas evolution subsided. The oil bath temp was then raised to 160-165° C. and held at this temperature for the duration of the reaction. As the oil bath temp was being raised a stream of nitrogen (2.5 L/min) was passed over the reaction solution, this nitrogen flow would be maintained for the duration of the reaction. The reaction was complete in 16 h and 20 min. The reaction was allowed to cool and then had added to it isopropanol, the addition of which would precipitate 2-(4-aminophenyl)-1,3-benzoxazol-5-amine, presumably as a methanesulfonic acid salt, as well as some impurities. The crude solid was isolated via filtration, washed with additional isopropanol, and then allowed to dry overnight under a stream of nitrogen. 16.11 g of crude product isolated as a brown solid wet with isopropanol and methanesulfonic acid and containing some impurities.

### Example 3

This example demonstrates the synthesis of 4,4′-[1,3]thiazolo[4,5-f][1,3]benzothiazole-2,6-diyldianiline from 2,5-diaminobenzene-1,4-dithiol dihydrochloride and p-aminobenzoic acid in methanesulfonic acid.

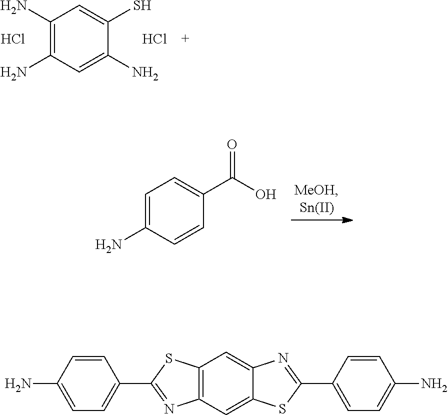

In a nitrogen filled glovebox to a 100 mL round bottomed Schlenk flask equipped with a magnetic stir bar was added 172 mg of SnCl₂ dihydrate, 4.00 g 2,5-diaminobenzene-1,4-dithiol dihydrochloride, 4.32 g p-aminobenzoic acid, and 27.1 mL of methanesulfonic acid. Methanesulfonic acid addition would cause HCl outgassing which was captured via a sodium carbonate bath. The flask was purged with nitrogen and then lowered into a hot oil bath whose temp was 75° C. The oil bath temp was held at 75° C. until HCl gas evolution subsided. The oil bath temp was then raised to 165° C. and held at this temperature for the duration of the reaction. As the oil bath temp was being raised a stream of nitrogen (2.5 L/min) was passed over the reaction solution, this nitrogen flow would be maintained for the duration of the reaction. The reaction was allowed 10 h before being taken off heat and allowed to cool. The cooled reaction had added to it water, the addition of which precipitated 4,4′-[1,3]thiazolo[4,5-f][1,3]benzothiazole-2,6-diyldianiline along with some reaction intermediates and impurities (8.439 g of solid when dried of water). The crude solid was isolated via filtration, resuspended in water, and the solution brought to pH 7 by adding concentrated ammonium hydroxide. Solids are again collected via filtration, they are a mixture of 4,4′-[1,3]thiazolo[4,5-f][1,3]benzothiazole-2,6-diyldianiline along with some reaction intermediates and impurities. Pure 4,4′-[1,3]thiazolo[4,5-f][1,3]benzothiazole-2,6-diyldianiline can be obtained by crystallizing the aforementioned neutralized solids in N-methylpyrrolidinone.

### Example 4

This example demonstrates the synthesis of 2-(4-aminophenyl)-5-amino-benzimidazole from 1,2,4-triaminobenzene dihydrochloride and 4-aminobenzoic acid in methanesulfonic acid, with p-xylene as an azeotroping solvent.

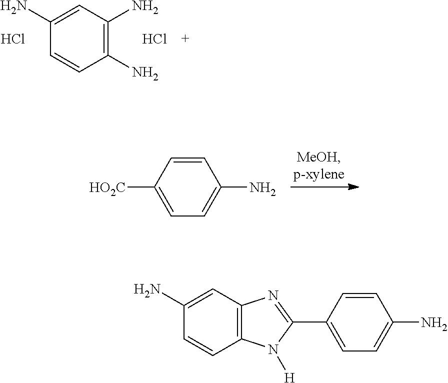

To a round bottomed flask equipped with a mechanical stirrer, a graduated Dean-Stark trap, and a reflux condenser and was added 350 mg of SnCl₂ dihydrate, 6.57 g 1,2,4-triaminobenzene dihydrochloride, and 4.59 g 4-aminobenzoic acid. Added to the flask were 30 mL of methanesulfonic acid and 50 mL of p-xylene. The receiving arm of the Dean Stark trap was prefilled with p-xylene. The reaction was heated at reflux temperature, under nitrogen, for 8.25 h. To the reaction mixture was added 90 mL water. The mixture was then heated to 85° C. and 25 mL of 12.1M HCl was added. Crystalline solids appeared on cooling. The solids were filtered and washed with fresh 12.1M HCl. A small second crop was obtained on the addition of additional HCl to the filtrate. The solids were combined and dissolved in 70 mL hot water. After the solution cooled to 30° C., the mixture was brought to pH 9 using ammonium hydroxide. A light green solid (5.86 g) was isolated by filtration. The structure of the product was confirmed by ¹H NMR and Mass Spectrometry.

### Example 5

This example demonstrates the synthesis of 2-(4-aminophenyl)-5-amino-benzimidazole from 1,2,4-triaminobenzene dihydrochloride and 4-aminobenzoic acid in methanesulfonic acid, with octane as an azeotroping solvent.

To a flask equipped with a Dean-Stark trap filled to capacity with octane, was added 12 mL of octane, 4 mL of methanesulfonic acid, 60 mg of SnCl₂ dihydrate, 1.31 g 1,2,4-triaminobenzene dihydrochloride, and 0.919 g 4-aminobenzoic acid. The solution was slowly heated to reflux and continued heating at reflux for 17 hours, at which time 2.0 mL of water was added to hydrolyze a small amount of amide which formed, the amide being the 1:1 condensation product of the desired product and 4-aminobenzoic acid. The reaction was removed entirely from heat 1 hour after the addition of water.

Upon cooling the methanesulfonic acid layer solidified into a light blue solid mass. The remaining octane was decanted from the solids and the solids added to 16 mL of water which was then brought to 85° C. with added activated carbon. After ten minutes of stirring at 85° C. with the activated carbon, the solution was filtered and washed with 2 mL of water. The filtrate was brought back to 85° C. and 12.1 M HCl was added to it, causing precipitation of colorless product which continued upon solution cooling. The solids were collected through filtration and washed with 12.1 M HCl and isopropanol then allowed to air dry (1.51 g recovered, 68% yield from this crop). A second crop of hydrochloride crystallized from the filtrate as it was being diluted with HCl and isopropanol (193 mg, which combined with crop 1 brought the overall yield to 76%).

### Example 6

This example demonstrates the synthesis of 4,4′-(1,5-dihydroimidazo[4,5-f]benzimidazole-2,6-diyl)dianiline tetramesylate from 4-aminobenzoic acid and 1,2,4,5-tetraminobenzene tetrahydrochloride in methanesulfonic acid with p-xylene as an azeotroping solvent.

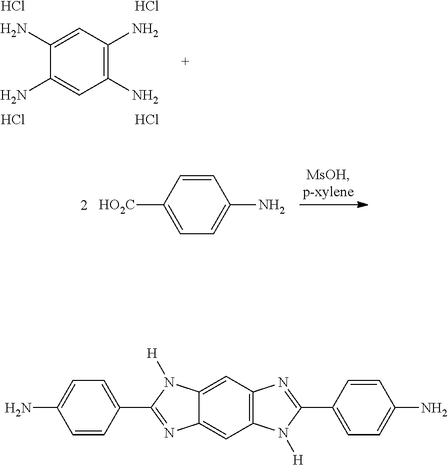

The preparation of 4,4′-(1,5-dihydroimidazo[4,5-f]benzimidazole-2,6-diyl)dianiline tetramesylate was accomplished following the same general procedure described in Example 4.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value.

In this specification, unless explicitly stated otherwise or indicated to the contrary by the context of usage, where an embodiment of the subject matter hereof is stated or described as comprising, including, containing, having, being composed of or being constituted by or of certain features or elements, one or more features or elements in addition to those explicitly stated or described may be present in the embodiment. An alternative embodiment of the subject matter hereof, however, may be stated or described as consisting essentially of certain features or elements, in which embodiment features or elements that would materially alter the principle of operation or the distinguishing characteristics of the embodiment are not present therein. A further alternative embodiment of the subject matter hereof may be stated or described as consisting of certain features or elements, in which embodiment, or in insubstantial variations thereof, only the features or elements specifically stated or described are present.

In this specification, unless explicitly stated otherwise or indicated to the contrary by the context of usage, amounts, sizes, ranges, formulations, parameters, and other quantities and characteristics recited herein, particularly when modified by the term “about”, may but need not be exact, and may also be approximate and/or larger or smaller (as desired) than stated, reflecting tolerances, conversion factors, rounding off, measurement error and the like, as well as the inclusion within a stated value of those values outside it that have, within the context of this invention, functional and/or operable equivalence to the stated value.

What is claimed is:

1. A process for preparing a compound that is represented by the structure of the following Formula (I) 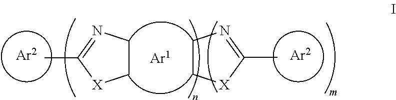
comprising the steps:
c) forming a reaction mixture comprising
i) a stoichiometric mixture of an aromatic amine component which is an ortho-substituted aromatic amine represented by the structure of the following Formula (II), 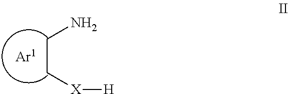
or an acid salt of such an amine;
and an aromatic acid component represented by the structure of the following Formula (III), and 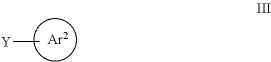
ii) a solvent comprising an organic sulfonic acid represented by the structure of the following Formula (IV); and 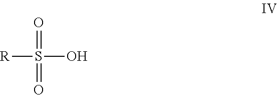
d) heating the reaction mixture at a temperature below the decomposition temperature of the organic sulfonic acid, for a time sufficient to effect a reaction to produce the composition that is represented by the structure of Formula (I);
wherein:
each Ar¹ is a substituted or unsubstituted, monocyclic or polycyclic, arylene or heteroarylene group;
each Ar² is a substituted or unsubstituted, monocyclic or polycyclic, aryl or heteroaryl group;
n=1 or 2 and m=0 or 1;
each X is independently NH, O, or S;
each Y is independently COOH, COOR′, COHal, CONH₂, or CN; and
R and each R′ are each independently an alkyl, aryl, alkaryl, or aralkyl group and Hal is a halogen atom.

2. The process according to claim 1 wherein the organic sulfonic acid is methanesulfonic acid or p-toluenesulfonic acid.

3. The process according to claim 1 wherein the aromatic amine component is: 1,2-diaminobenzene, 1,2,4-triaminobenzene; 1,2,4,5-tetraminobenzene; 1,2-diaminopyridine, 1,2,4-triaminopyridine, 2,3,5,6-tetraminopyridine; 2,4-diaminophenol; 2-aminophenol; 2-aminothiophenol; 2,5-diaminobenzene-1,4-dithiol; an acid salt of any of the preceding; or a mixture of at least two of these or of their acid salts.

4. The process according to claim 1 wherein the aromatic acid component is: benzoic acid, a halo-substituted benzoic acid, an alkyl-substituted benzoic acid, 4-aminobenzoic acid, 3-aminobenzoic acid, 2-aminobenzoic acid, terephthalic acid, isophthalic acid, benzene-1,2-dicarboxylic acid, terephthaloyl chloride, isophthaloyl chloride, benzonitrile, benzamide, an alkyl benzoate, or a mixture of at least two of these.

5. The process according to claim 1 wherein Ar¹ is 3-amino-o-phenylene, C₆H₃NH₂, 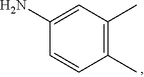
Ar² is 4-aminophenyl, C₆H₄NH₂, 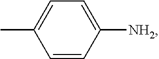
Y is COOH, X is NH, R is CH₃, and the compound of Formula (I) thereby produced is 2-(4-aminophenyl)-5-amino-benzimidazole,

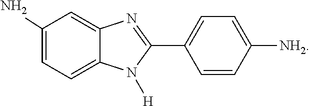 [SMILES: Nc1ccc(-c2nc3cc(N)ccc3[nH]2)cc1]

6. The process according to claim 1, wherein Ar¹ is 3-amino-o-phenylene, C₆H₃NH₂, 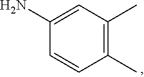
Ar² is p-C₆H₄COOH, 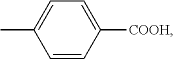
Y is COOH, X is NH, R is CH₃, and the compound of Formula (I) thereby produced is

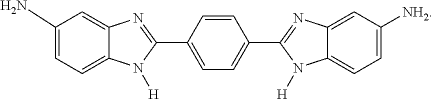 [SMILES: Nc1ccc2[nH]c(-c3ccc(-c4nc5cc(N)ccc5[nH]4)cc3)nc2c1]

7. The process according to claim 1 wherein Ar¹ is 3,4-diamino-o-phenylene,
C₆H₂ (NH₂)₂, 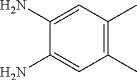
Ar² is 4-aminophenyl (C₆H₄NH₂), 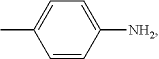
Y is COOH, X is NH, R is CH₃, and the compound of Formula (I) thereby produced is

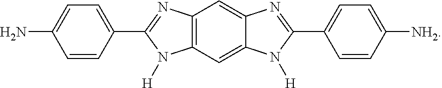 [SMILES: Nc1ccc(-c2nc3cc4nc(-c5ccc(N)cc5)[nH]c4cc3[nH]2)cc1]

8. The process according to claim 1 wherein the reaction mixture further comprises a reducing agent, wherein said reducing agent is capable of reducing oxidation byproducts at the pH of the reaction mixture.

9. The process according to claim 8 wherein the reducing agent is Sn(0), Sn(II), Cr(II), Mn(II), Fe(0), Fe(II), Co(0), Co(II), Ni(0), Ni(II), Cu(0), Cu(I), Zn(0), Mg(0), or a mixture thereof.

10. The process according to claim 1 wherein aromatic amine component and aromatic acid component together are present in the reaction mixture at 1 to 30 weight percent, based on the combined weight of aromatic amine, aromatic acid, and organic sulfonic acid.

11. The process according to claim 10 wherein aromatic amine component and aromatic acid component together are present in the reaction mixture at 15 to 17 weight percent, based on the combined weight of aromatic amine, aromatic acid, and organic sulfonic acid.

12. The process according to claim 11 wherein the organic sulfonic acid is methanesulfonic acid or p-toluenesulfonic acid, Y is COOH, Ar¹ is 3-amino-o-phenylene, and Ar² is 4-aminophenyl; further comprising Sn or Sn(II) as a reducing agent.

13. The process according to claim 1 wherein the reaction mixture further comprises a solvent that is immiscible with the organic sulfonic acid under reaction conditions, boils between 100° C. and 200° C., and forms an azeotrope with water produced by the reaction; wherein the process further comprises the step of removing said azeotrope from the reaction mixture by distillation.

14. The process according to claim 13 wherein the solvent is octane, a xylene, toluene, a chlorobenzene, or 1-butanol.
